(19) United States
(12) Patent Application Publication
(10) Pub. No.: US 2016/0175292 A1
(43) Pub. Date: Jun. 23, 2016

(54) Antioxidants Having Aromatic Structures Reacting with Superoxide

(71) Applicant: James Tour, Bellaire, TX (US)

(72) Inventors: James Tour, Bellaire, TX (US); Redwan Huq, Houston, TX (US); Jason A. Mann, Houston, TX (US); Almaz Jalilov, Houston, TX (US); Lizanne Nilewski, Houston, TX (US); Tom Kent, Houston, TX (US); Christine Beeton, Pearland, TX (US)

(21) Appl. No.: 14/978,448

(22) Filed: Dec. 22, 2015

Related U.S. Application Data
(60) Provisional application No. 62/095,624, filed on Dec. 22, 2014.

(51) Int. Cl. A61K 31/4375

(52) U.S. Cl. CPC A61K 31/4375

(57) ABSTRACT

Disclosed is a method of treating diseases which are: reactive oxygen species mediated, ischemic or reperfusion-related, or T-cell mediated, including autoimmune diseases. The method is administering a therapeutically effective amount of a formulation wherein the active ingredient includes non-phenolic aromatic structures that are electron deficient and are capable of converting the superoxide radical to O₂; and/or of converting superoxide radical to oxygen and hydrogen peroxide, or pharmaceutically acceptable salts of said structures. Also disclosed is a method of diagnosing and treating such diseases and conditions.

### STATEMENT REGARDING FEDERALLY SPONSORED RESEARCH

This invention was made with government support under Grant Nos. W81XWH-08-2-0143 and W81XWH-08-2-0141 awarded by the U.S. Department of Defense; and Grant No. R21 DK093802, awarded by the National Institutes of Health. The government has certain rights in the invention.

### BACKGROUND

Antioxidant compounds and antioxidant materials, including polyethylene-glycol functionalized hydrophilic carbon clusters (PEG-HCCs), U.S. Pat. No. 8,916,606 (incorporated by reference), can be useful in treating a number of diseases and conditions. As mimetics of the activity of PEG-HCCs, such antioxidants can serve as scavengers of superoxide or other oxidants, making them potentially useful as immunomodulators, and therapeutics for oxidative stress-related conditions. Such antioxidants can be useful as modulators of reactive oxygen species and trauma or injury or ischemic-related diseases, and also function as inhibitors of T cell activation, and thus in treatment of T-cell mediated diseases, including autoimmune diseases such as Multiple Sclerosis and Rheumatoid Arthritis. See WO 2015/034930 (incorporated by reference).

Certain antioxidants which are electron-deficient aromatic structures—but not phenolic aromatics—are capable of converting superoxide to O₂; and/or of converting superoxide to oxygen and hydrogen peroxide. These antioxidants, including such diimide antioxidants can be derivatized to make them more suitable for therapy by, for example, reducing their toxicity, increasing their lipophilicity (thus potentially facilitating their crossing the blood brain barrier, their bioavailability or their time in circulation), allowing them to target, e.g., mitochondria, ligands, hormones, cell surface or other receptors or enzymes. They can also be linked to fluorescent dyes to allow visualization of the constructs and/or targeted cells or tissues by fluorescence or NIR imaging in theranostic applications; or they can be linked to DTPA[Gd] or DOTA[Gd], to allow the constructs and/or targeted cells or tissues to be studied by MRI imaging in theranostic (therapy and diagnostic) applications.

### SUMMARY

Non-phenolic aromatic structures that are electron deficient and are capable of converting the superoxide radical to O₂ which is formally called dioxygen and more commonly “oxygen”; and/or of converting superoxide radical to oxygen and hydrogen peroxide. These are useful as antioxidants for treating T-cell mediated diseases, including autoimmune diseases, and for treating acute injuries where superoxide is overexpressed, such as in traumatic brain injury, stroke, ischemia/reperfusion such as in organ transplant, as well as in chronic injuries where superoxide is overexpressed such as in neuropathy. Such electron-deficient aromatic structures include, e.g., perylene derivatives including perylene diimides (PDI) and derivatives of perylene diimides, which have high photostabilities and absorption/emission wavelengths in biologically relevant ranges, and are further useful as theranostic (therapy and diagnostic) probes. Further, such electron-deficient aromatic structures include, e.g., coronene and its derivatives; naphthalene derivatives and naphthalene diimides (NDI) and its derivatives, as well as quinones and their derivatives, and generally fused aromatic molecules with electronic withdrawing groups that render them electron deficient relative to polyaromatics that do not bear the electronic withdrawing groups. The electron withdrawing groups on the polyaromatics can include imides, amides, esters, carboxyls, ketones, and aldehydes, (carbonyl compounds in general), as well as nitro, cyano, sulfonyl, sulfate, and halogens such as fluoro, chloro, bromo and iodo groups, and include Such electron deficient aromatics include the following structures:

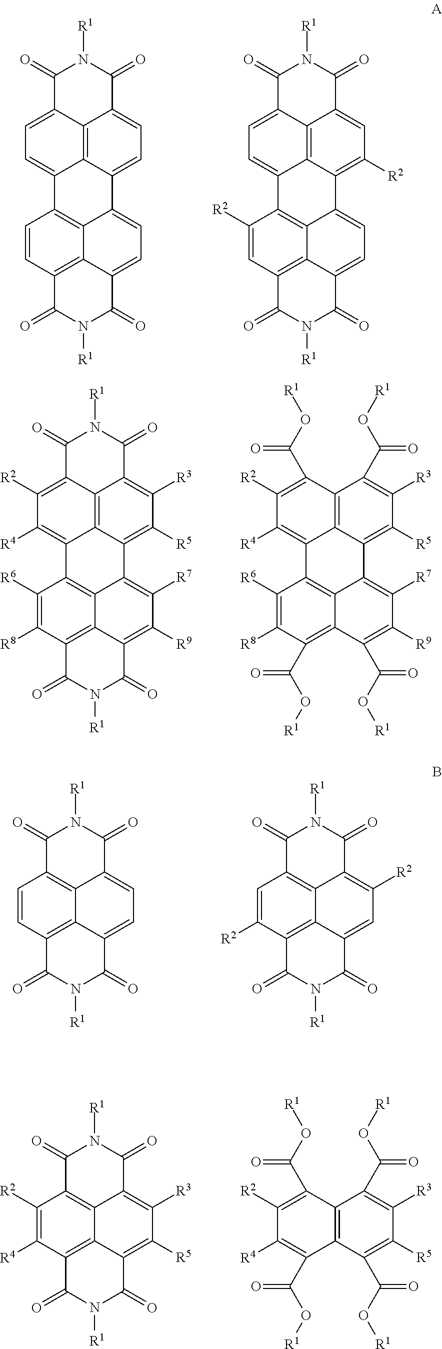

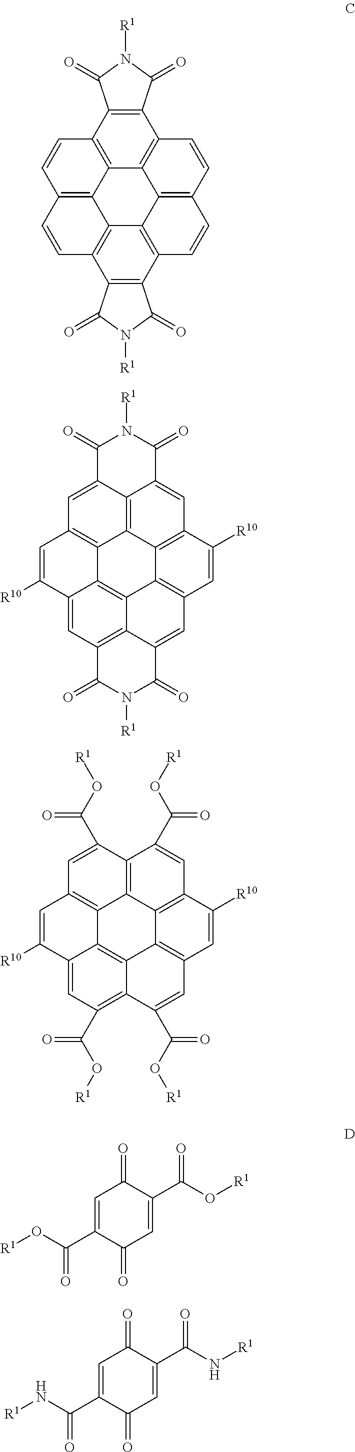

In the structures above, compounds in row A are perylenes, compounds in row B are naphthalenes, and compounds in row C are coronenes; and row D shows benzoquinone di-esters, and benzoquinone di-amides, respectively.

R1 can be any of: H, polyethylene glycol (PEG); PEG-OMe, PEG-O-alkyl; PEG-O-aryl; PEG-OR; PEG-R; PEG-N₃; PEG-alkenyl; PEG-alkynyl; PEG-dye; PEG-DTPA[M]; PEG-DOTA[M]; PEG-adamantyl; PEG-CO₂H; aryl; heteroaryl; alkyl; alkenyl; alkynyl; heteroalkyl; R-PPh₃+; R—N₃; R-alkenyl; R-alkynyl; R—CO₂H; NH—R; O-alkyl; O-aryl; and, perfluoroalkyl. These are more generally groups that can aid in water solubility.

R2-R9 can be any electron withdrawing group, including: halogens, including Cl, Br, F, I; CF₃; R—Cl; R—Br; R—I; R—CF₃; carbonyl; nitrile; amide; imide; cyano; carboxylic acid; carboxylate; ester; ketone; aldehyde; nitro; cyano; fluoro, chloro; bromo; iodo; sulfonate; sulfoxide, sulfone, alkyl sulfonate, sulfonic acid, alkyl sulfonates, aryl; arylamino; arylimido; arylcyano; fused/extended aryl ring systems; heteroaryl; alkyl; alkenyl; alkynyl; hetroalkyl; acyl; NO₂; NH—R; O—R; SH—R; O-alkyl; O-aryl; and, acyl-R;

R10 can be any group or combination of groups set forth in R1 to R9;

R can be any compatible functional groups; and

M can be any and all compatible metals.

Similar structures and substitutions can be made on other non-phenolic aromatic structures that are electron deficient and are capable of converting superoxide to oxygen; and/or of converting superoxide to oxygen and hydrogen peroxide. Such other non-phenolic ring structures, include aromatic and heterocylic rings, including pyrrole, thiophene, furan, pyrimidine, purine, isoquinoline, quinoline, benzofuran, indole, and oxazole. In some cases, further appending of aromatic rings to the above structures will also render electron deficient and are capable of converting superoxide to oxygen; and/or of converting superoxide to oxygen and hydrogen peroxide.

Some embodiments of the invention relate to antioxidants which are derivatives, analogs, functionalized or modified versions of perylenes, naphthalenes, coronenes and quinones for use in therapeutic applications, and more particularly, for inhibition of T cell proliferation in treating T-cell mediated diseases including autoimmune diseases. These derivatives, analogs, functionalized or modified versions of perylenes, naphthalenes, cpronenes and quinones include polyethylene glycol-functionalized (PEGylated) derivatives thereof, diimide derivatives thereof, and combinations thereof, including PEGylated perylene diimides (PEG-PDIs), PEGylated naphthalene diimides (PEG-NDIs), quinone derivatives (e.g., PEGylated quinones), perylenediimide (PDI) derivatives; naphthalenediimide (NDI) derivatives; quinine imide derivatives; coronene imide derivatives and combinations thereof. The antioxidants can be further modified to allow fluorescence or NIR imaging of their targets (or in the case of perylenediimides which are themselves fluorescent, increase the signal) by adding fluorescent dyes, or perylenediimide groups; or, to allow MRI imaging of their targets, by, for example, adding DTPA[Gd] or DOTA[Gd] groups.

In some embodiments of the structures depicted above, where the R1 and/or R2 groups are halogens, the position and number of the halogen groups in the structures can vary to tune the electronic properties of the carbon core, and to induce twisting, thereby further modulating the electronic properties.

The invention includes administering therapeutically effective amounts of such non-phenolic aromatic structures that are electron deficient to patients to treat T-cell mediated diseases, including autoimmune diseases. Exemplary autoimmune diseases include rheumatoid arthritis, multiple sclerosis, rheumatoid arthritis, reactive arthritis, ankylosing spondylitis, systemic lupus erythematosus, glomerulonephritis, psoriasis, scleroderma, alopecia aerata, type 1 diabetes mellitus, celiac sprue disease, colitis, pernicious anemia, encephalomyelitis, vasculitis, thyroiditis, Grave's disease, Addison's disease, Sjogren's syndrome, antiphospholipid syndrome, autoimmune cardiomyopathy, autoimmune hemolytic anemia, autoimmune hepatitis, autoimmune inner ear disease, autoimmune lymphoproliferative disorder, autoimmune peripheral neuropathy, pancreatitis, polyendocrine syndrome, thrombocytopenic purpura, uveitis, Behcet's disease, narcolepsy, myositis, polychondritis, asthma, chronic obstructive pulmonary disease, graft-versus-host disease, and chronic graft rejection. In other applications, ischemic tissue generates superoxide upon reperfusion, so compounds of the invention are also useful for ischemia or reperfusion conditions, including, trauma e.g. traumatic brain injury, ischemia, anoxic encephalopathy, hypoxic or ischemic encephalopathy, cerebrovascular dysfunction, hemorrhagic shock, hypoxia, hypotension, Alzheimer's disease, Parkinson's disease, multiple sclerosis, amyotrophic lateral sclerosis (ALS or Lou Gehrig's disease), liver disease, non-alcoholic fatty liver disease, diabetes, stroke, inflammation, spinal cord injury (SCI), central nervous system injury (CNSI) or neuropathy, organ transplantation (treatment of the organ or the patient) and combinations thereof. Furthermore, the methods and compositions of the present disclosure may be used to treat oxidative stress with minimal toxicity and side effects.

The invention includes methods for the use of therapeutically effective amounts of one or more of such non-phenolic aromatic structures that are electron deficient, in the manufacture of a medicament or dosage form. Such medicaments, formulations and dosage forms include, for example, topical delivery forms and formulations (which may be particularly useful for treating skin lesions caused by psoriasis or other autoimmune conditions). Preferably, the medicament, formulation or dosage form is a foam, cream, spray or gel. The dosage form can also be administered orally, transdermally, sublingually, intra-rectally, intra-nasally and/or parenterally.

In another aspect, the invention includes an article of manufacture comprising a vessel containing a therapeutically effective amount of one or more pharmaceutically acceptable non-phenolic aromatic structures that are electron deficient, and instructions for use. Such instructions may include instructions regarding topical administration for treatment of a subject having psoriatic plaques or lesions, or for oral administration or administration is by intramuscular, intradermal, intravenous, subcutaneous, intraos seous, intraperitoneal, intrathecal, epidural, intracardiac, intraarticular, intracavernous, or intravitreal injection. Instructions may include instructions for administration, adverse events, side effects, interactions with other medications, and other warning and cautionary notes.

The invention also includes pharmaceutically acceptable salts of the non-phenolic aromatic structures that are electron deficient. Such salts possess the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

In theranostic applications of the invention, the first step is to determine if a compound of the invention binds to or interacts with the superoxide radical in vivo. This can be done by labeling (e.g., with a with a radioactive isotope) of such a compound, and then determining if it appears in increased concentrations at in vivo sites where the superoxide radical is expected. If such is the case, additional dosages can be administered for treating the T-cell mediated disease or condition.

The compounds of the invention may have asymmetric centers, chiral axes, and chiral planes, and occur as racemates, racemic mixtures, and as individual diastereomers, with all possible isomers, enantiomers, cis isomers, trans isomers, conformational isomers, and mixtures thereof, including optical isomers, being included in the present invention. In addition, the compounds disclosed herein may exist as tautomers and both tautomeric forms are intended to be encompassed by the scope of the invention, even though only one tautomeric structure may be depicted.

These and other aspects of the present inventions, which are not limited to or by the information in this Brief Summary, are provided below.

### BRIEF DESCRIPTION OF THE DRAWINGS

FIG. 1 shows the synthesis of naphthalene (Rows V & X) and perylene diimides (Rows W & Y), from their respective anhydrides; as well as the synthesis of certain derivatives of naphthalene and perylene diimides, such that, the A, B or C group (box on lower right) links at its amino end, and A, B, or C (without the NH₂ group) becomes the R group in the compounds produced (right-hand side) of Rows X and Y.

FIG. 2A schematically shows the reactions for making certain derivatized perylenes and naphthalenes from anhydrides thereof. Row A shows making of nitrate derivatives of perylene. The di-brominated perylene B can be used as a starting material in the synthesis of a number of derivatives, including the alkyne derivative C, which can in turn be used to synthesize alkane derivatives D and E. The naphthalene derivative in the last row can be used to synthesize the ether-derivative (F) or the alkyl-derivative (G).

In FIG. 2B, compounds in Row A are ether derivatives of perylenes. Compound B is a triphenylphosphine derivative of naphthalene diimide, and Compound C is a triphenylphosphine derivative of perylene diimide. Compound D is a polyethylene glycol (repeated n times) derivative of naphthalene diimide and Compound D is a polyethylene glycol (repeated n times) derivative of perylene diimide.

FIG. 3 shows the synthesis of coronene and certain derivatives thereof, which are also antioxidant compounds of the invention. R in FIG. 3 is preferably a group to help make it more hydrophilic and water soluble; e.g., PEG, carboxylic acid; polyvinyl alcohol, salts of acids, including sodium salts of carboxylic acid, sulfates and sulfonates.

FIG. 4 shows the synthesis of polyethylene glycol diimide derivatives of naphthalene (Compound A) and perylene (Compound B) from their respective anhydrides. Compounds C and D are respectively naphthalene and perylene derivative starting materials for synthesis.

FIG. 5A shows the synthesis of ether derivatives of naphthalene (Compound A) and alkyl derivatives of naphthalene (Compound B), from naphthalene anhydride.

FIG. 5B shows the synthesis of a perylene diimide derivative (Compound C) and brominated derivatives of perylene (Compounds D and E), from perylene diimide.

FIG. 5C shows synthesis of an alkylated perylene derivative (Compound G) and a coronene diimide derivative (Compound F).

FIG. 5D shows synthesis of a PEGyated perylene derivative (Compound H), from perylene anhydride.

FIG. 6 shows the synthesis of certain polyethylene glycol derivatives of perylene (Compound A), and ester derivatives of perylene (Compounds B and C), from perylene anhydride. R in FIG. 6 can be salts of acids; carboxylates; or PEGylated esters, or any of: H, polyethylene glycol (PEG); PEG-OMe, PEG-O-alkyl; PEG-O-aryl; PEG-OR; PEG-R; PEG-N₃; PEG-alkenyl; PEG-alkynyl; PEG-dye; PEG-DTPA[M]; PEG-DOTA[M]; PEG-adamantyl; PEG-CO₂H; aryl; heteroaryl; alkyl; alkenyl; alkynyl; heteroalkyl; R-PPh₃+; R—N₃; R-alkenyl; R-alkynyl; R—CO₂H; NH—R; O-alkyl; O-aryl; and, perfluoroalkyl;

FIG. 7A shows structures of some known naphthalene, perylene and quinone inhibitory compounds. Compounds A and B are respectively naphthalene and perylene-based inhibitors of Pinl; where Pinl regulates cell cycle progression and is required for the assembly, folding, and transport of cellular proteins. Compound C is a State-3 inhibitor, which binds to a hypoxiainducible factor and selectively induces cancer cell death. Compound D (Amonafide) and E (Elinafide) are topoisomerase inhibitors: DNA intercalators that induce DNA strand breaks and prevent unwinding of DNA by Topoisomerase. Compound F is a water-soluble perylene diimide derivative found to be nontoxic, and having a twisted, non-planar core.

FIG. 7B shows two compounds, A and B, both of which are water-soluble perylene diimide derivative found to be nontoxic, and having a twisted, non-planar core.

FIG. 7C shows synthesis of derivatized carotenes (Compound A) from perylene anhydride; and synthesis of derivatized naphthalenes (Compounds B and C), from brominated naphthalene anhydrides.

FIG. 7D, Compound A is a Histone Deacetylase inhibitor; Compound B is the toxic product of P450 metabolism of benzopyrene; Compounds C and E are mutagenic secondary metabolites of Alternaria molds; Compounds D, F, G and H are mutagenic products of P450 metabolism of benzoperylene; Compound I is a telomerase inhibitor.

FIG. 8 shows structural formulas for several examples of compounds of the invention. Where Row A shows perylene derivatives including perylene diimides (both compounds at the right in row A); Row B shows. naphthalene derivatives including naphthalene diimides (both compounds at the right in row B); Row C shows coronene derivatives including coronene diimide (middle compound in Row C); Row D shows quinones derivatives. The same compounds are listed in the same order in the Summary and in the claims below.

FIG. 9 shows that EG₈-PDI (LNA30) and TEG-NDI (LNA20) exhibit an inhibitory effect on T-cell proliferation.

FIG. 10 shows that EG₈-PDI (LNA30) and TEG-NDI (LNA20) exhibit an inhibitory effect on T-cell proliferation.

FIG. 11 shows that PEG-HCCs and p-benzoquinones (pBQ) show near complete consumption of superoxide.

FIG. 12 is a cyclic voltometry (“CV”) plot of 50 mM PBS (pH=7.4) bare glassy carbon (GC) working electrode and with modified perylene tetracarboxylic anhydride (PTCA) and HCC. (Scan rate: 100 mV/s).

FIG. 13 is CV plot of N₂ saturated DMSO solution of 0.2 mM PEG-NDI.

FIG. 14 is CV plot of O₂ saturated DMSO solution of 0.2 mM PEG-NDI. (0.1 M TBAP, Scan rate: 200 mV/s).

FIG. 15 shows the UV-Vis spectroscopic changes of PEG-PDI solution in DMSO in the presence of KO₂ under N2 atmosphere.

FIG. 16 shows the UV-Vis spectroscopic changes of PEG-NDI solution in DMSO in the presence of KO₂ under N2 atmosphere.

### DETAILED DESCRIPTION

The term “alkyl” as used herein refers to a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” as used herein refers to a substituting univalent group derived by conceptual removal of one hydrogen atom from a straight or branched-chain acyclic unsaturated hydrocarbon containing at least one carbon-carbon double bond (i.e., —CH═CH₂, —CH═CHCH₃, C═C(CH₃)₂, —CH₂CH═CH₂, etc.).

The term “alkynyl” as used herein refers to a substituting univalent group derived by conceptual removal of one hydrogen atom from a straight or branched-chain acyclic unsaturated hydrocarbon containing at least one carbon-carbon triple bond (i.e., —C≡CH, —C≡CCH₃, —C≡CCH(CH₃)₂, —CH₂C≡CH, etc.).

The term “aryl” as used herein refers to a substituting univalent group derived by conceptual removal of one hydrogen atom from a cyclic aromatic hydrocarbon.

The term “aryloxy” as used herein refers to an aryl group with a bridging oxygen atom, such as phenoxy (—OC₆H₅), or benzoxy (—OCH₂C₆H₅).

“Arylamino” means an aryl group with a bridging amine function such as —NHCH₂C₆H₅.

“Arylamido” means an aryl group with a bridging amide group such as —(C═O)NHCH₂C₆H₅, or an aryl group with a imide group such as —(C═O)₂NCH₂C₆H_{5.}

The term “cycloalkyl” as used herein refers to a substituting univalent group derived by conceptual removal of one hydrogen atom from a saturated monocyclic hydrocarbon (e.g., cyclopropyl, cyclobutyl, cyclopentyl, cyclohexyl, or cycloheptyl).

The term “heteroaryl” as used herein refers to a substituting univalent group derived by the conceptual removal of one hydrogen atom from an aromatic ring system containing one or more heteroatoms selected from N, O, or S. Examples of heteroaryl groups include, but are not limited to, pyrrolyl, furyl, thienyl, imidazolyl, pyrazolyl, oxazolyl, isoxazolyl, thiazolyl, pyridyl, pyrimidinyl, pyrazinyl, benzimidazolyl, indolyl, and purinyl. Heteraryl substituents can be attached at a carbon atom or through the heteroatom. Examples of moncyclic heteroaryl groups include pyrrolyl, furyl, thienyl, pyrazolyl, oxazolyl, isoxazolyl, thiazolyl, and pyridyl. Examples of bicyclic heteroaryl groups include pyrimidinyl, pyrazinyl, benzimidazolyl, indolyl, and purinyl. Individual rings may have 5 or 6 atoms. Thus, this includes a 4-membered moncyclic heteroaryl group and a 5-memebered monocylcic heteroaryl group. It also includes a bicyclic heteroaryl group having one 5-membered ring and one 6-membered ring, and a bicyclic heteroaryl group having two 6-membered rings.

1. Making Active Ingredients

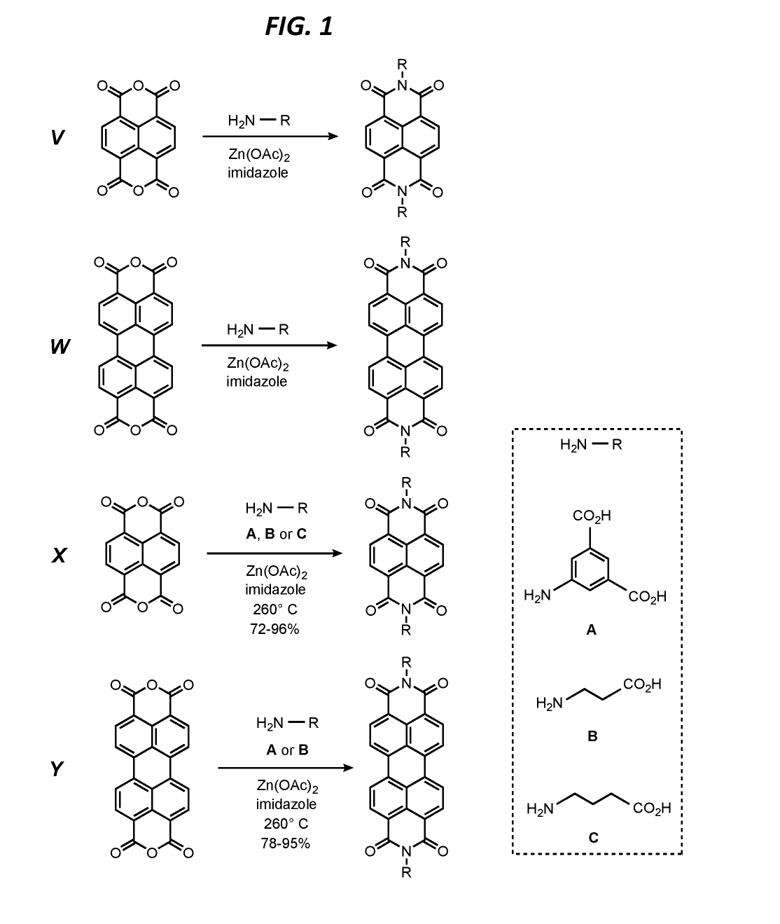

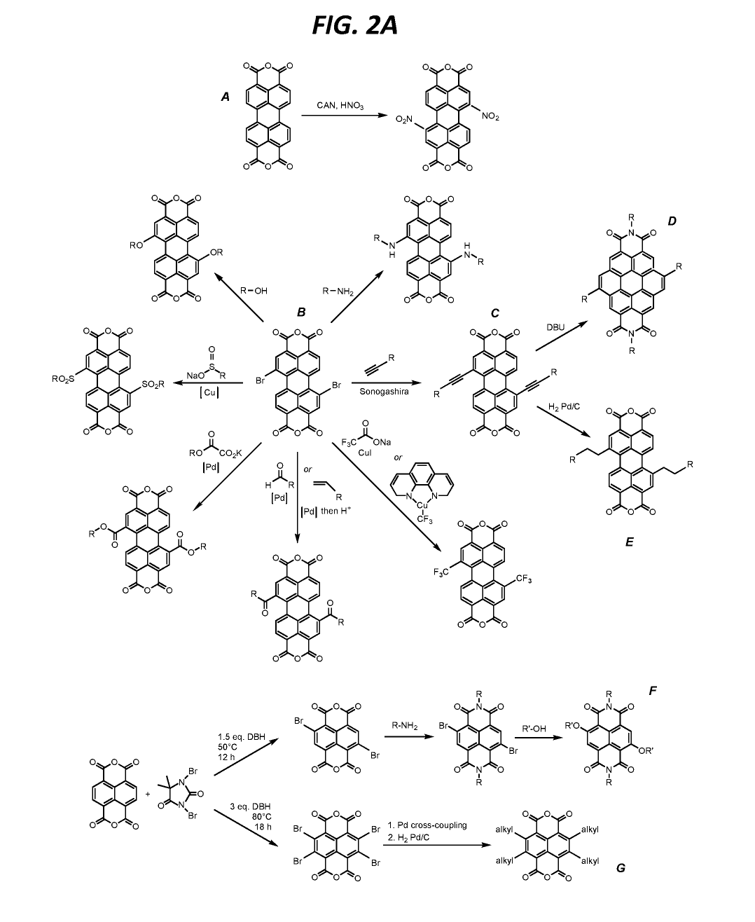

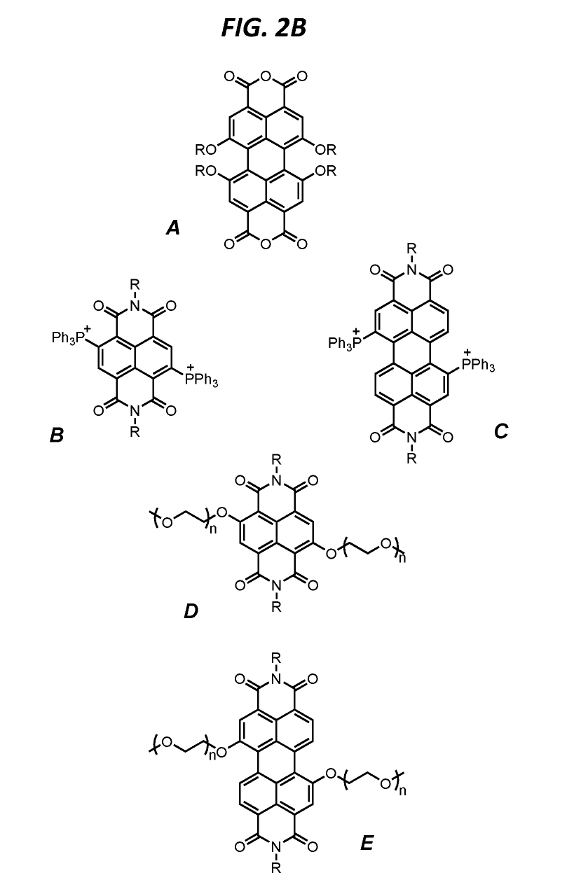

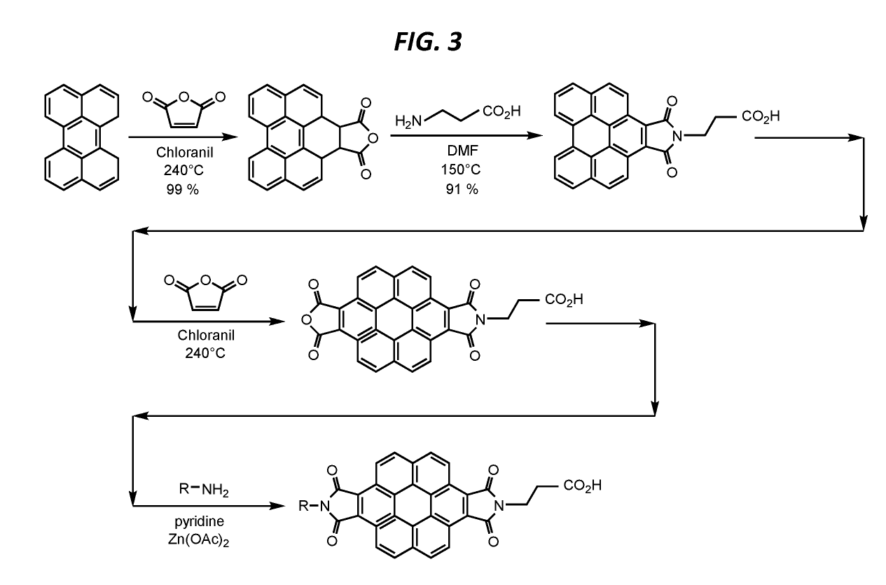

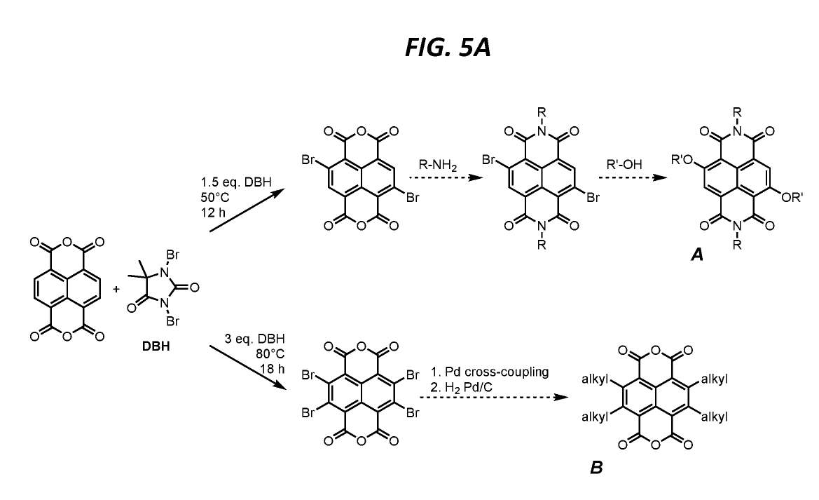

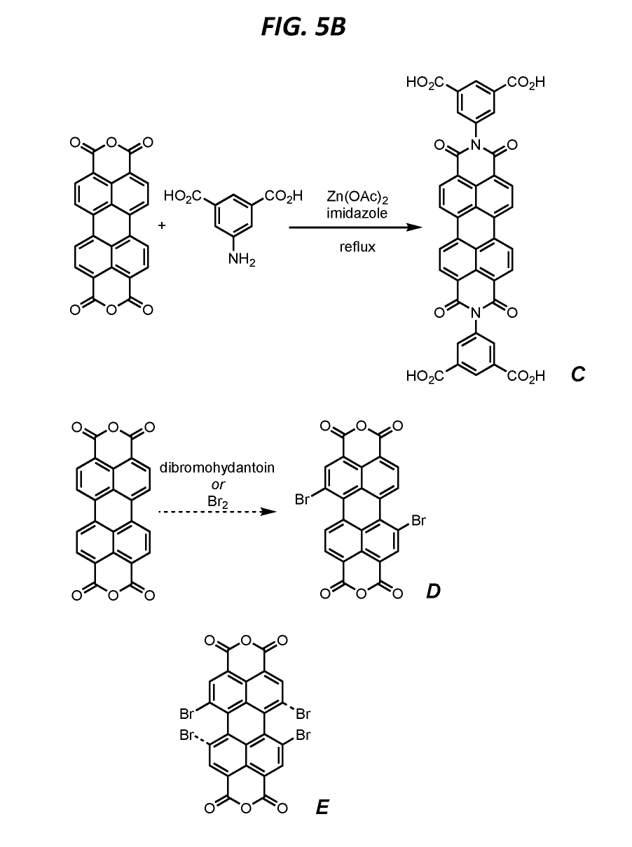

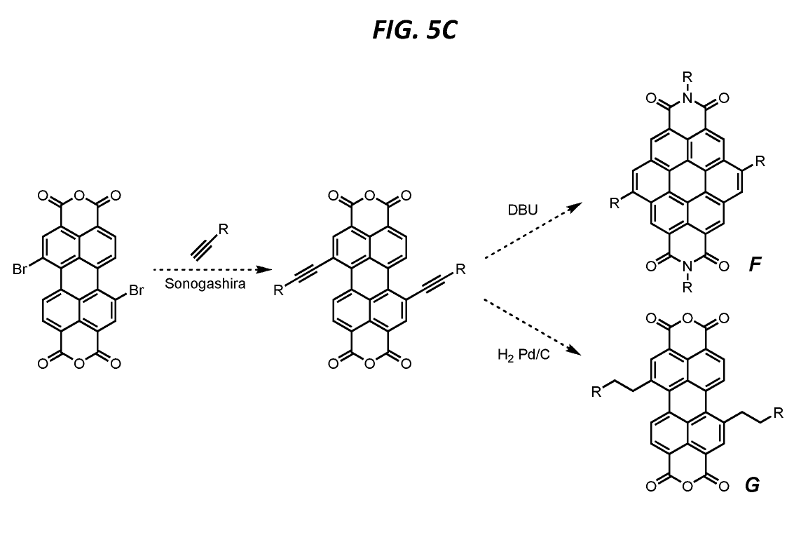

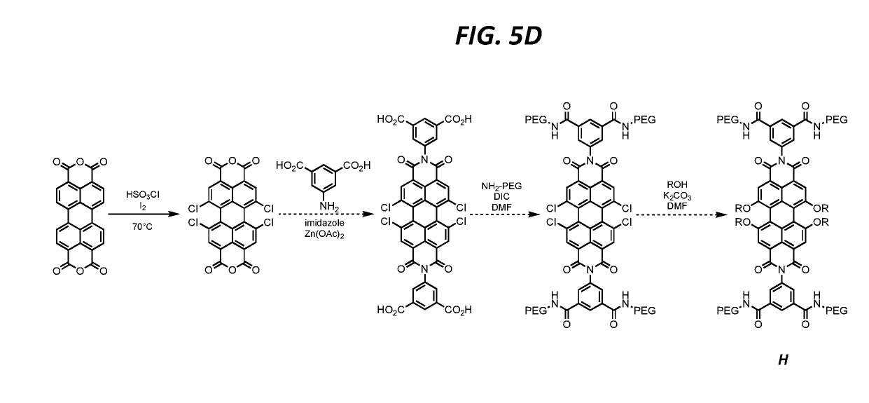

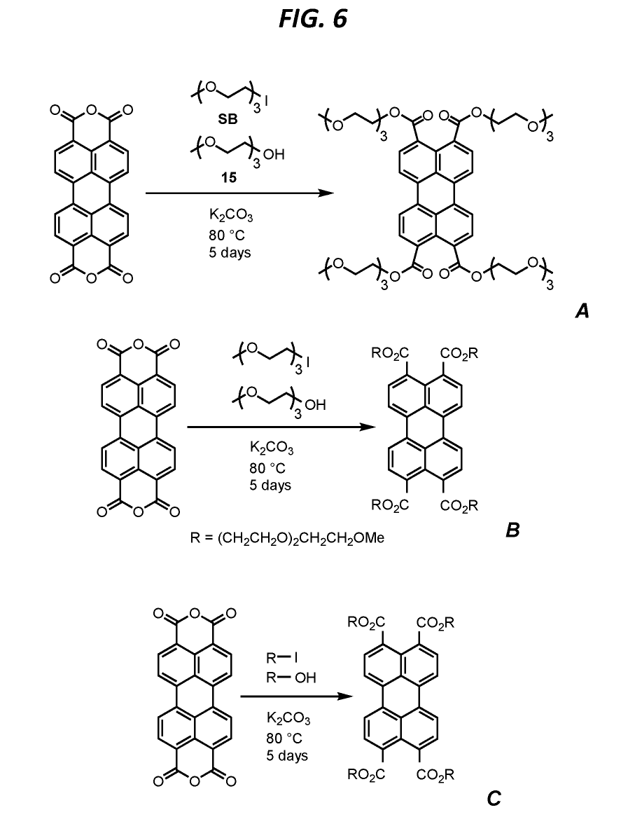

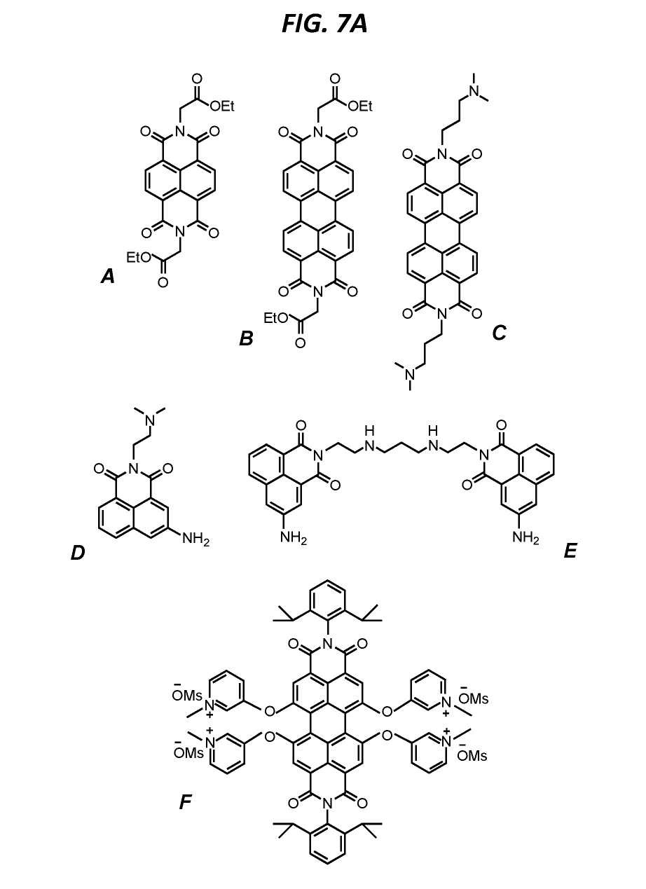

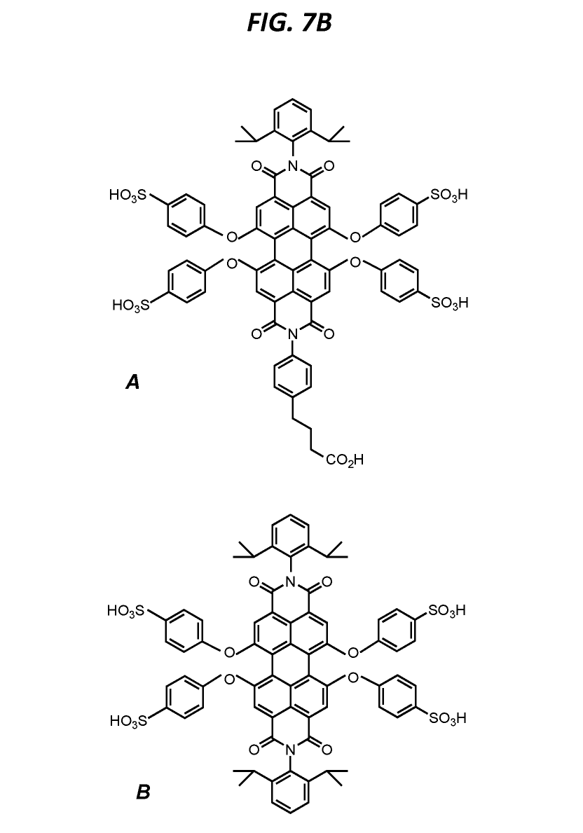

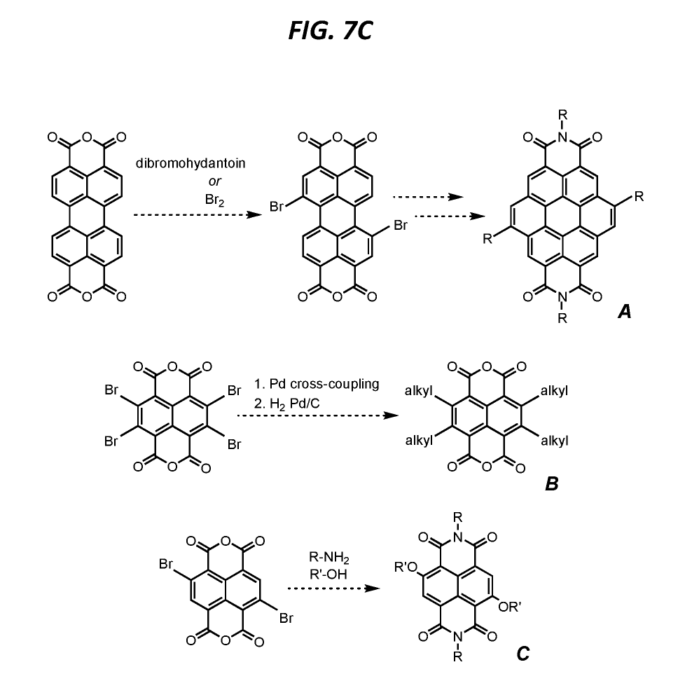

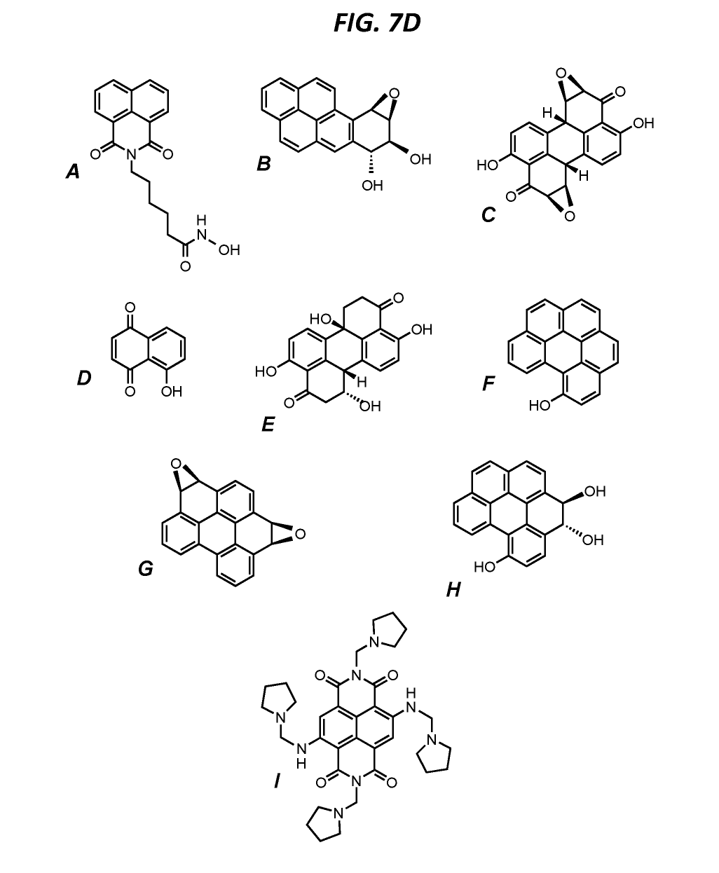

FIGS. 1-7 shows the synthesis schemes for a number of the compounds of the invention. Other compounds within the scope of the invention are manufactured by similar processes, if similar, or by other processes well-known by those skilled in the art. A number of starting materials and other compounds not within the scope of the invention are also shown in these figures for reference in synthesis. Common derivatives would include the appending of further fused aromatic rings to the structure.

2. Making Formulations of Active Ingredients for Administration

For preparing solid compositions such as tablets, the principal active ingredient is mixed with a pharmaceutical excipient to form a solid pre-formulation composition containing a homogeneous mixture of a compound of the invention. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation is then subdivided into unit dosage forms of the type described above containing from. The tablets or pills of the invention may be coated or otherwise compounded to provide a dosage form affording the advantage of prolonged action.

For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid forms in which the novel compositions of the invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil; as well as elixirs and similar pharmaceutical vehicles.

Compositions for inhalation or insufflation include solutions and suspensions in pharmaceutically acceptable, aqueous or organic solvents, or, mixtures thereof, and powders. The liquid or solid compositions may contain suitable pharmaceutically acceptable excipients as described supra. Preferably the compositions are suitable for oral or nasal respiratory administration, for local or systemic effect.

Compositions in preferably pharmaceutically-acceptable solvents may be nebulized by use of inert or atmospheric-like gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

3. Support for the Use of the Compounds of the Invention in Therapy

A. PEG-HCCs in Treatment of T-Cell Mediated and Autoimmune Diseases

The use of hydrophilic carbon cluster (also known as ultra-short single-walled carbon nanotubes: “US-SWNTs”) functionalized with poly(ethylene glycol) as superoxide radical quenchers, along with substantial evidence of their efficacy against T-cell mediated and autoimmune disease, is described in International Application No. WO 2015/034930 (incorporated by reference), entitled “Treatment of Inflammatory Diseases by Carbon Materials.” To review the experiments described therein, it was demonstrated that the hydrophilic carbon clusters functionalized with polyethylene glycol (PEG-HCC), are preferentially taken up by T cells, and the failure to take up PEG-HCC leaves major functions of macrophages intact—indicating a likelihood of not inducing generalized immunosuppression when administered in vivo. Incubation of rat splenocytes with PEG-HCC exhibited an increased PEG-HCC signal upon cell permeabilization, indicating that these compounds were internalized and not just bound to the cell surface. Moreover, such an effect was more apparent in CD3+ cells, suggesting that PEG-HCCs were preferentially internalized by T cells.

An evaluation of the uptake of PEG-HCCs by other rat immune cells, such as CD3⁻ negative splenocytes was undertaken, and the permeabilization of macrophages (CD3⁻B220⁻Ly6G⁻CD103⁻ CD11b⁺), B cells (CD3⁻B220⁺), NK cells (CD3⁻CD161a⁺), dendritic cells (CD3⁻B220⁻Ly6G⁻ CD103⁺) and neutrophils (CD3⁻B220⁻Ly6G⁺) did not increase PEG-HCC signals, indicating that T cells selectively uptake PEG-HCCs. In vivo studies (where PEG-HCCs were injected into rats subcutaneously) confirmed that PEG-HCCs preferentially enter T cells over macrophages, B cells, NK cells, dendritic cells and neutrophils.

Incubation of primary GFP-transduced ovalbumin-specific rat T cells (CD4⁺CCR7⁻ CD45RC⁻ Kv1.3^{high}) with PEG-HCCs, followed by stimulation of the cells with ovalbumin, demonstrated a dose-dependent reduction in both intracellular SO levels and T cell proliferation. However, the decrease in T cell proliferation was not due to the presence of PEG, which alone was not sufficient to induce an inhibitory response. In addition, washing away excess PEG-HCCs and immediately stimulating the cells did not alter the effect on proliferation, confirming that PEG-HCCs need to be internalized to alter T cell activity. However, stimulating the cells after 6 hours rescued the inhibitory effect on proliferation. This result is in alignment with the kinetics of molecule loss and suggests that PEG-HCCs have a reversible effect on T cell activity.

The reduction in T cell proliferation was demonstrated not to be due to cytotoxicity of the PEG-HCCs, because cell viability of T cells treated with the molecules was unaltered. Also studied were the effects of PEG-HCCs on the production of pro-inflammatory cytokines in T cells stimulated by ovalbumin. A 30% reduction in the levels of interleukin (IL)-2 and interferon (IFN)-gamma was observed. Other results indicated that PEG-HCCs do not affect the production of T cell chemo-attractants by macrophages, and did not affect the migration of T cells. Other results indicated that PEG-HCCs do not modify antigen processing and presentation by macrophages.

Effects of PEG-HCCs on animal disease models that are mediated by T cells was examined. A single subcutaneous injection of 2 mg/kg of PEG-HCCs in the ears of rats decreased inflammation against ovalbumin challenge.

EAE is a T cell mediated inflammatory autoimmune process of the central nervous system (CNS) that resembles the human demyelinating disease multiple sclerosis (MS). PEG-HCCs effect on rats with myelin basic protein-induced EAE was tested. Subcutaneous treatment of rats with 2 mg/kg of PEG-HCCs every three days starting at the onset of disease signs significantly reduced clinical scores. Histologic analysis of spinal cords isolated from EAE rats at the peak of disease revealed a decrease in inflammatory foci, indicating decreased infiltration of immune cells into the spinal cord.

Other results showed that PEG-HCCs can reduce the number of lesions to the bloodbrain barrier in a model of multiple sclerosis in rats. Pristane-induced arthritis, an animal model of rheumatoid arthritis, was induced and monitored in rats. It was found that the administration of PEG-HCCs every four days starting at the onset of clinical signs significantly reduced disease severity. PEG-HCCs also showed a trend towards reducing R-EAE clinical scores during the relapsing phase of EAE disease. R-EAE was induced in a small cohort of DA rats (n=9 rats; split into 2 treatment groups) and a prevention trial with PEG-HCCs was conducted. PEG-HCCs displayed a minor inhibitory effect on the first episode of disease.

These results, particularly those from the rat models that PEG-HCCs lead to a reduction in DTH inflammation and in lesions, as well as in EAE scores and immune infiltration into the spinal cord, as well as reducing RA severity in the rat model; indicates that because the compounds of the invention, like PEG-HCCs, quench superoxide (see below), the compounds of the invention are also likely to be useful in treating T cell mediated diseases, and autoimmune diseases.

B. Compounds of the Invention Exhibiting Superoxide Quenching and other Antioxidant Properties

Certain compounds of the invention, particularly, PEGylated and modified perylene diimides (PEG-PDIs) and naphthalene diimides (PEG-NDIs), and certain quinone derivatives have been demonstrated to exhibit antioxidant and superoxide quenching properties, similar to PEG-HCCs. These compounds also have structural features similar to that of the PEG-HCCs, making it even more likely that they are also useful in treating T cell mediated diseases, and autoimmune diseases. The compounds of the invention can be modified with a wide range of functional groups to modulate their electrochemical properties. For example, the aromatic carbon cores can be affixed with electron withdrawing groups thereby rendering the cores more electrophilic, and more likely to be reduced on reaction with superoxide. Conversely, the carbon cores can be made more electron rich with other known moieties to render them more able to donate electrons to superoxide, thereby generating hydrogen peroxide in the presence of two protons.

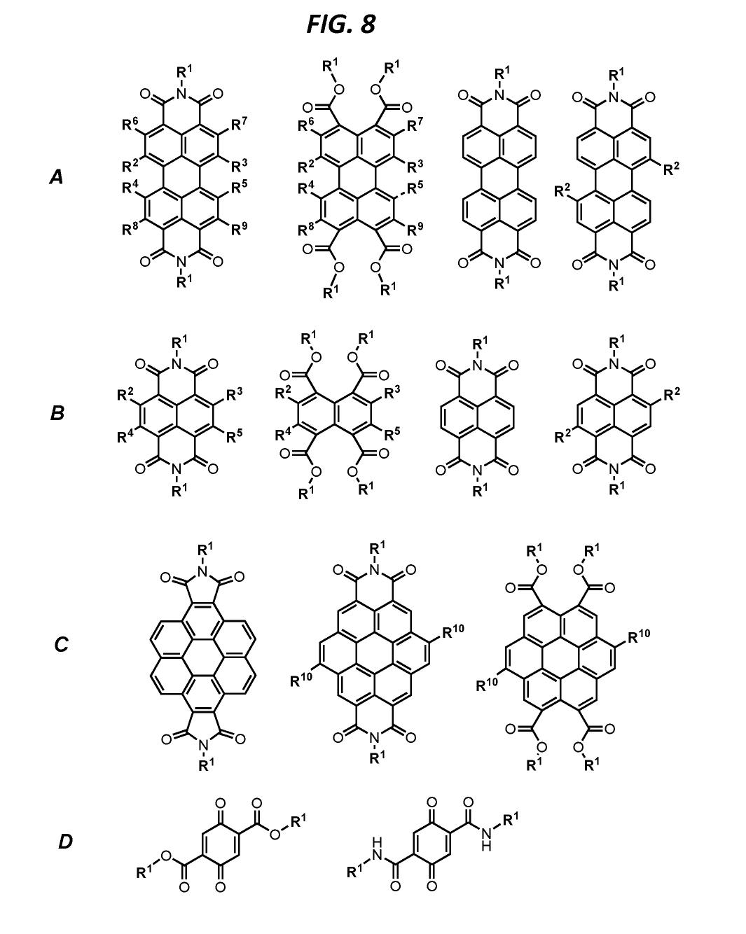

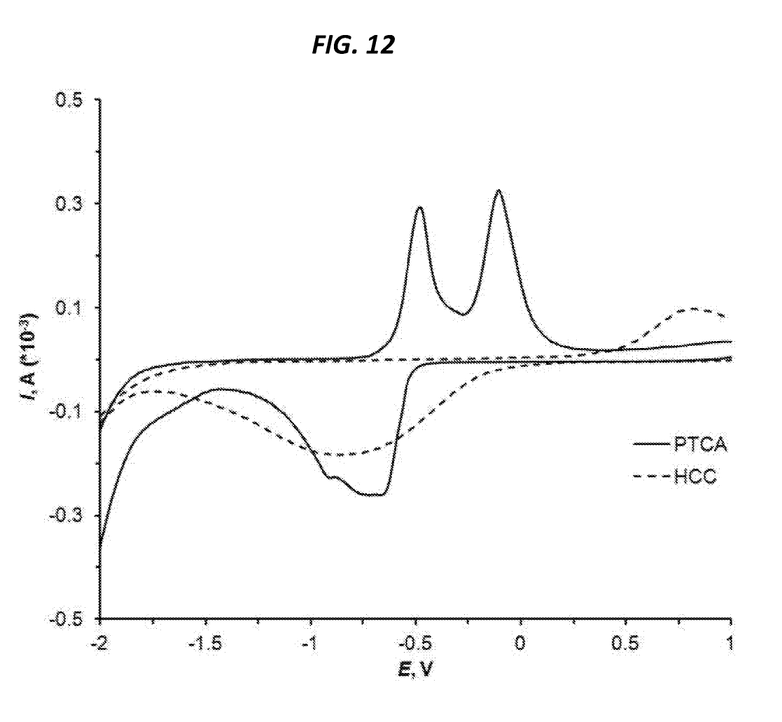

The triethyleneglycol-PDIs (TEG-PDIs) and TEG-NDIs have been analyzed electrochemically and show similar reduction potentials to the HCCs and PEG-HCCs. Electrochemical investigations of HCCs have placed its reduction at −0.7 V vs. Ag/AgCl, as shown by its broad peak in Plot A below. FIG. 12 also shows that perylene tetracarboxylic anhydride (FIG. 8, Row A, second compound from left, is the generic structure of a perylene tetracarboxylic anhydride) has a sharper peak at −0.7 V, indicating that HCC and perylene tetracarboxylic anhydride have about the same potential.

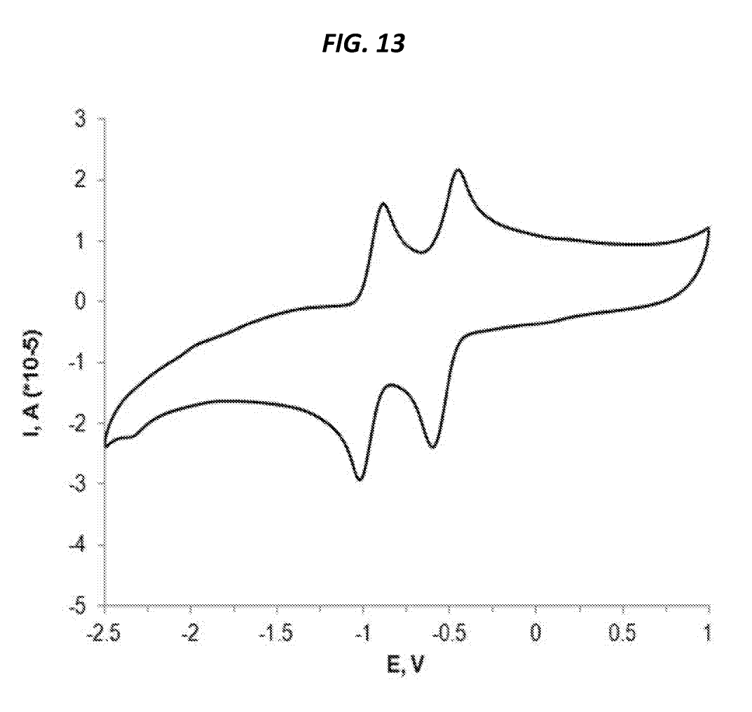

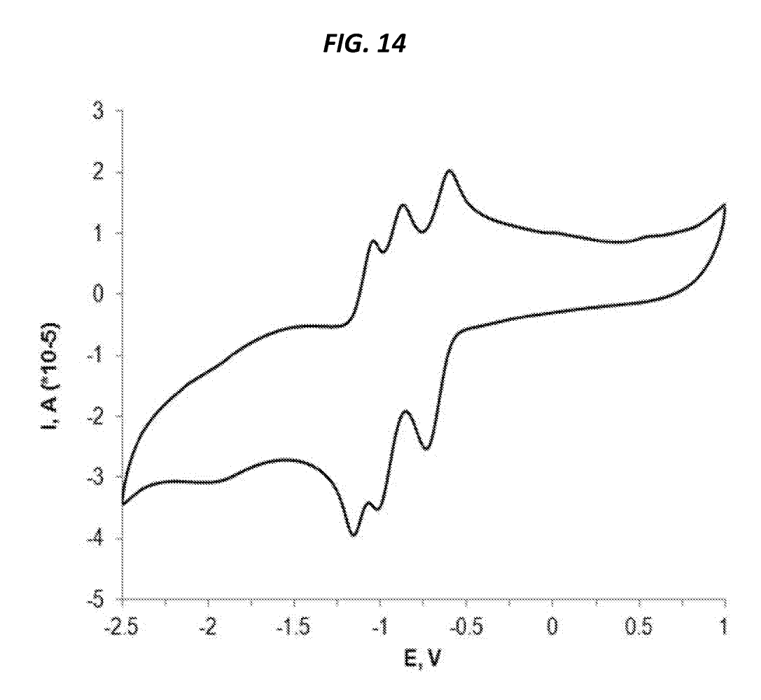

FIG. 13 shows that cyclic voltometry of N₂ saturated DMSO solution of 0.2 mM polyethylene glycol naphthane diimide (PEG-NDI), is similar to the O₂ saturated DMSO solution of 0.2 mM PEG-NDI (FIG. 14). FIG. 14 shows that the PEG-NDI with oxygen forms two peaks, with the oxygen peak in the middle.

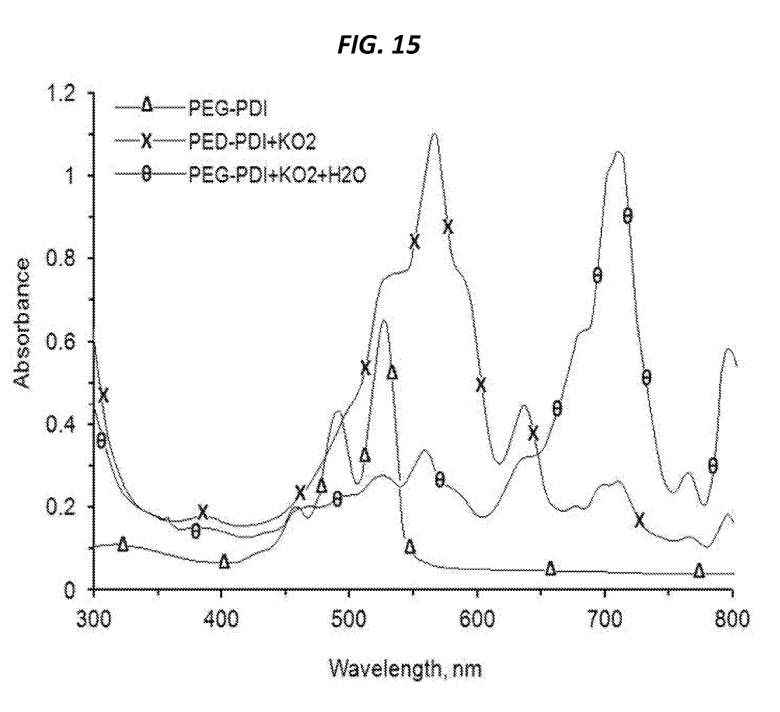

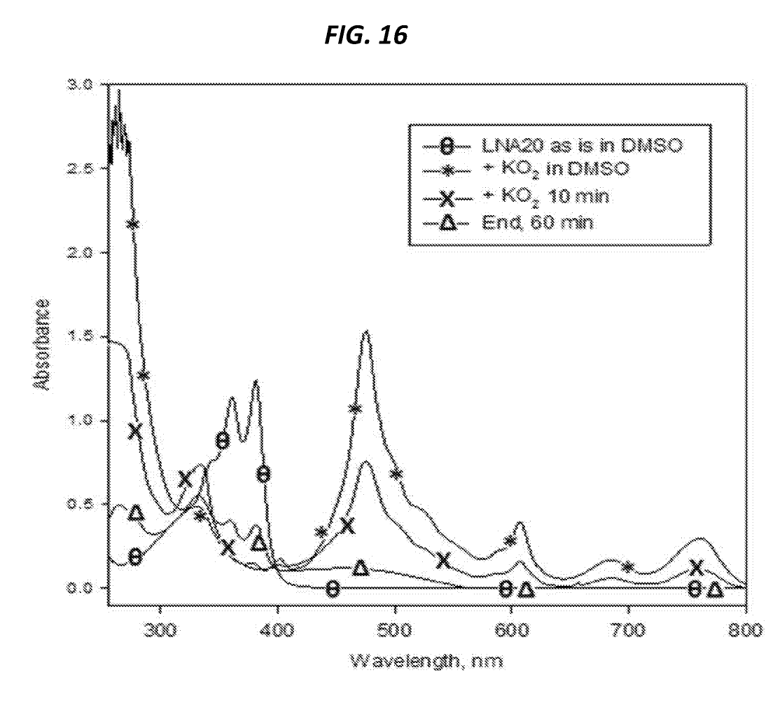

FIG. 15 shows that PEG-PDI reacts with KO₂ to produce a radical anion and then, again to produce a dianion. FIG. 16 shows that PEG-NDI reacts with KO₂ to produce a radical dianion which gradually decays over time, as superoxide reduces further to a dianion.

Accordingly, the triethyleneglycol-PDIs (TEG-PDIs) and TEG-NDIs have been analyzed electrochemically and shown to have similar reduction potentials to the HCCs and PEG-HCCs. Electrochemical investigations of HCCs have placed its reduction at —0.7 V vs. Ag/AgCl (Plot A). 3,4,9,10-Perylenediimide (PDI) derivatives also have redox potentials˜−0.6 V. 1,4,5,8-Naphthalenediimide (NDI) derivatives show discrete reduction steps in the same region (Plot B).

Additionally, in aqueous media, both PDI and NDI derivatives react with KO₂ to produce radical anions and dianions (Plots C and D) in a similar fashion to the proposed mechanism for PEG-HCC reaction with superoxide.

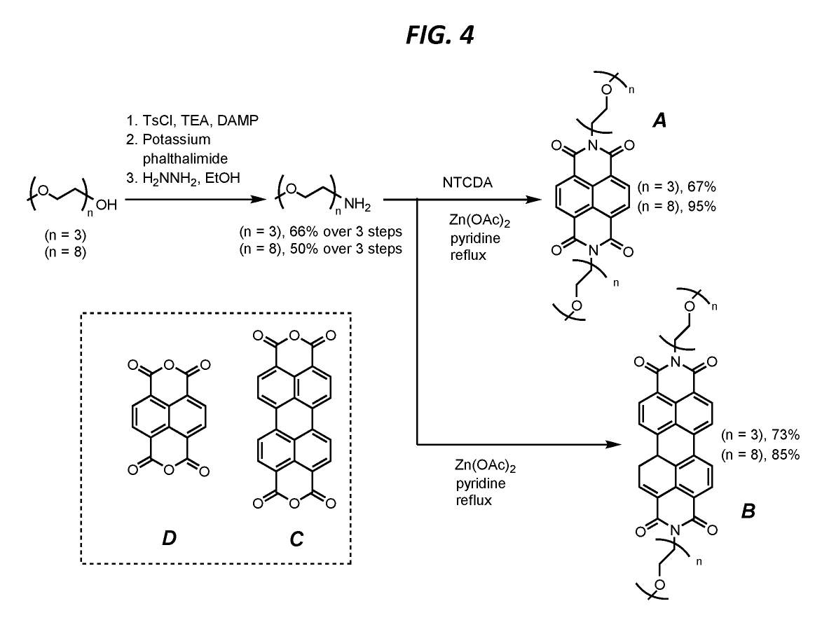

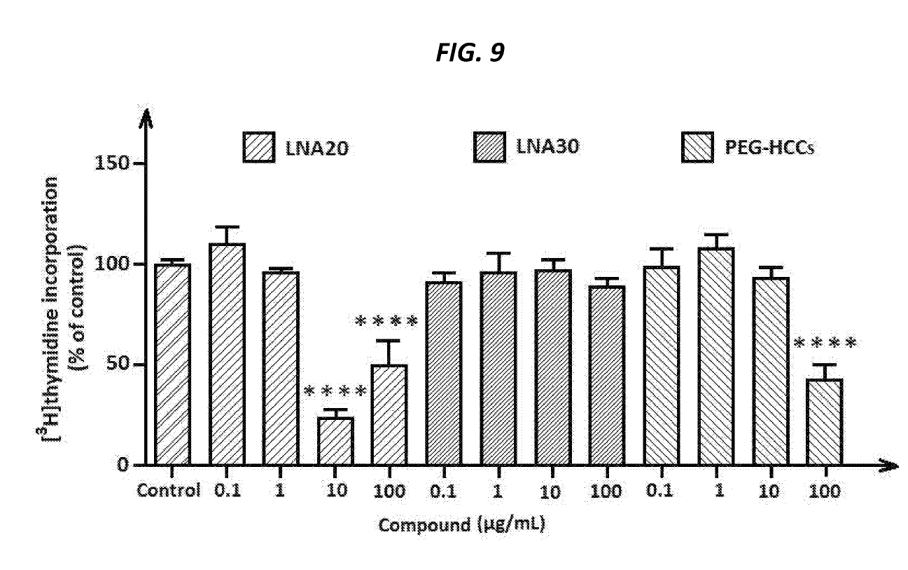

In vitro tests have demonstrated that EG₈-PDI (LNA30) and TEG-NDI (LNA20) exhibit an inhibitory effect on T-cell proliferation similar to that observed with PEG-HCCs (FIG. 9). In in vitro analysis of the effect of EG₃-NDI, labeled LNA20 (which is Compound A in FIG. 4 where n=3) and EG₈-PDI, labeled LNA30 (which is Compound B in FIG. 4 where n=8) on T cell proliferation, LNA20 showed inhibitory effects on T cell proliferation at concentrations above 10 μg/mL. See FIG. 9. This is similar to the activity observed with PEG-HCCs. EG8-PDI LNA30 (a perylene diimide) did not exhibit a reduction in T cell proliferation.

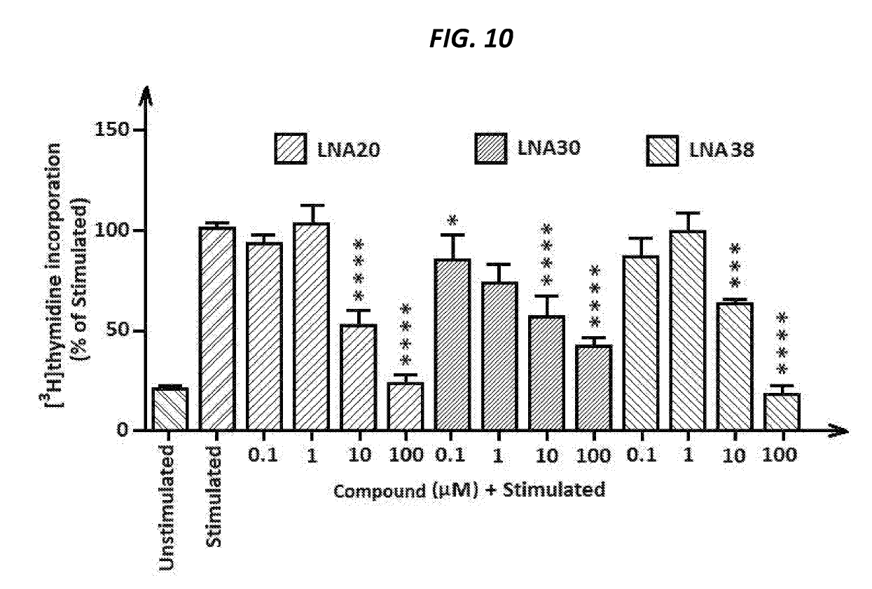

In vitro analysis of EG₃-NDI LNA20, EG₈-PDI LNA30, and EG₈-NDI, labeled LNA38 (which is Compound A in FIG. 4 where n=8) on T cell proliferation based on micromolar concentration was performed. 1 μg/mL ConA (mitogen) was used to stimulate rat T cells. All three small molecules inhibited T cell proliferation within the micromolar range to a similar degree, with LNA20 being the most potent and LNA30 being the least. See FIG. 10. Error bars represent variability between replicate wells. Unstimulated cells (left-hand bar) are shown as a negative control. Statistical analysis was performed between stimulated control cells without treatment (“Stimulated” bar) vs. various doses of the different compounds using a one-way ANOVA and Bonferonni post-hoc tests. *P<0.05, ***P<0.001, ****P<0.0001.

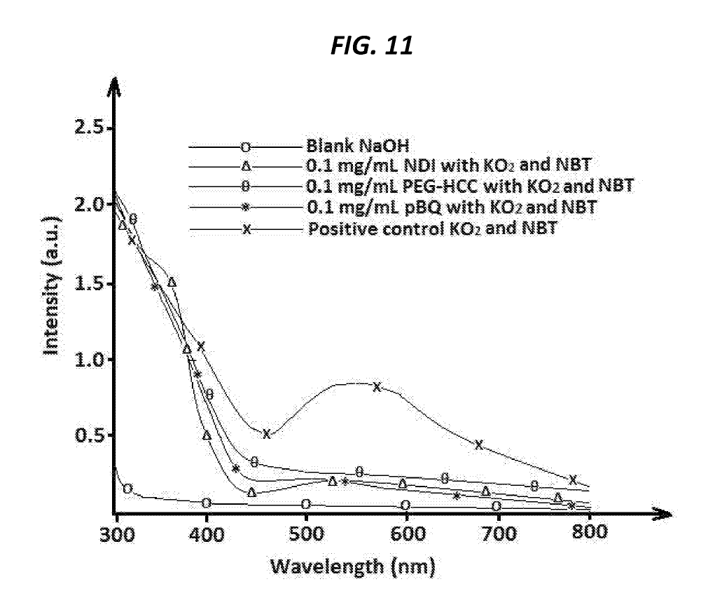

It was also demonstrated that LNA20 (grouped in FIG. 11) reacts with superoxide via a superoxide scavenging assay, using nitroblue tetrazolium (NBT) and KO₂. The antioxidant and buffer/solvent was added to a cuvette, followed by the KO₂ solution. After a set time (30 s, 60 s, 120 s, 300 s), NBT was added and the resulting mixture analyzed for the NBT diformazan (λₘₐₓ=560 nm) by UV-Vis spectroscopy (FIG. 11).

In FIG. 11, PEG-HCCs and p-benzoquinone (pBQ) were also run for comparison. The PEG-HCCs show near complete consumption of superoxide, while the positive control (without any antioxidant) showed significantly higher amounts of reduced NBT, and p-benzoquinone (pBQ) showed even less reduction of NBT, suggesting that the superoxide was nearly completely consumed by pBQ, even at a lower concentration than the HCCs. The NDI LNA20 (bis-methoxytriethylene glycol naphthalene diimide) also showed activity according to the test.

Compounds of the invention could be used in treating other T-cell mediated conditions, or conditions associated with excess superoxide, such as injury or ischemic reperfusion. Efficacy against all such diseases or conditions using the compounds of the invention can be determined in appropriate or recognized animal models.

4. Administration and Dosing Regimen of the Formulations

The formulations containing pharmaceutically active ingredients can be administered in any conventionally acceptable way including, but not limited to, intravenously, subcutaneously, intramuscularly, sublingually, topically, orally and via inhalation. Administration will vary with the pharmacokinetics and other properties of the drugs and the patient's condition.

The active ingredients are designed to treat certain T-cell mediated and autoimmune diseases. The amount of active ingredients alone to accomplish this is considered the therapeutically effective dose. The dosing schedule and amounts, i.e., the “dosing regimen,” will depend upon a variety of factors, including the stage of the disease or condition, the severity of the disease or condition, the severity of the adverse side effects, the general state of the patient's health, the patient's physical status, age and the like. In calculating the dosage regimen for a patient, the mode of administration is also taken into consideration. The dosing regimen must also take into consideration the pharmacokinetics, i.e., the rate of absorption, bioavailability, metabolism, clearance, and the like. Based on these values (which are determined in vitro, and in mammalian animal models and extrapolated to humans) the dosing regimen is projected for humans, and is then tested and further refined in clinical trials, in a conventional dose-finding study, as is well-known in the art.

The state of the art allows the clinician to determine the dosing regimen for each individual patient, depending on factors including administration route, disease stage, patient size, and patient level of wild-type MSP/VapB. For example, a physician may initially use escalating dosages, starting at a particular level, and then titrate the dosage at increments for each individual being treated based on their individual responses. Depending on the subject, the administration of the formulation is maintained for as specific period of time or for as long as needed to effectively treat the subject's symptoms or prevent their occurrence in the first place. In many autoimmune diseases, which are chronic, the treatment would normally be expected to continue for the patient's lifespan.

For instance, in some embodiments, the active ingredients can be administered at dosages that range from about 1 mg/kg of the subject's weight to about 5 mg/kg of the subject's weight, including at about 2 mg/kg of the subject's weight.

Based on the results of the experiments above, and particularly those in FIGS. 9, 10 and 11, it is clear that all three compounds (two derivatized perylene diimides and a derivatized naphthalene diimide) exhibit significant antioxidant activity at micromolar concentration. Accordingly, the starting dosages for a dose-finding study in humans with a pharmaceutical product with any of these or any related compounds would be in the micromolar range, or above. The dose-finding study could determine the suitable dosages in the manner well-known to those skilled in the art.

Another approach to finding dosages is to experiment with different dosages in animal disease models. Again, based on the in vitro studies above, the starting dosages in animals with any of the compounds from the in vitro studies, or with any related compounds, would also be in the micromolar range, or above. The results from the animal model experiments can be extrapolated to humans by, for example, multiplying the ratio of the weight difference by the doses(s) which had pharmacological effect in mice.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating any of the following diseases or conditions: reactive oxygen species mediated, ischemic or reperfusion-related, or T-cell mediated; comprising: administering a therapeutically effective amount of a formulation wherein the active ingredient includes non-phenolic aromatic structures that are electron deficient and are capable of converting the superoxide radical to O₂; and/or of converting superoxide radical to oxygen and hydrogen peroxide, or pharmaceutically acceptable salts of said structures.

2. The method of claim 1 wherein the non-phenolic aromatic structures include the following and their pharmaceutically acceptable salts: 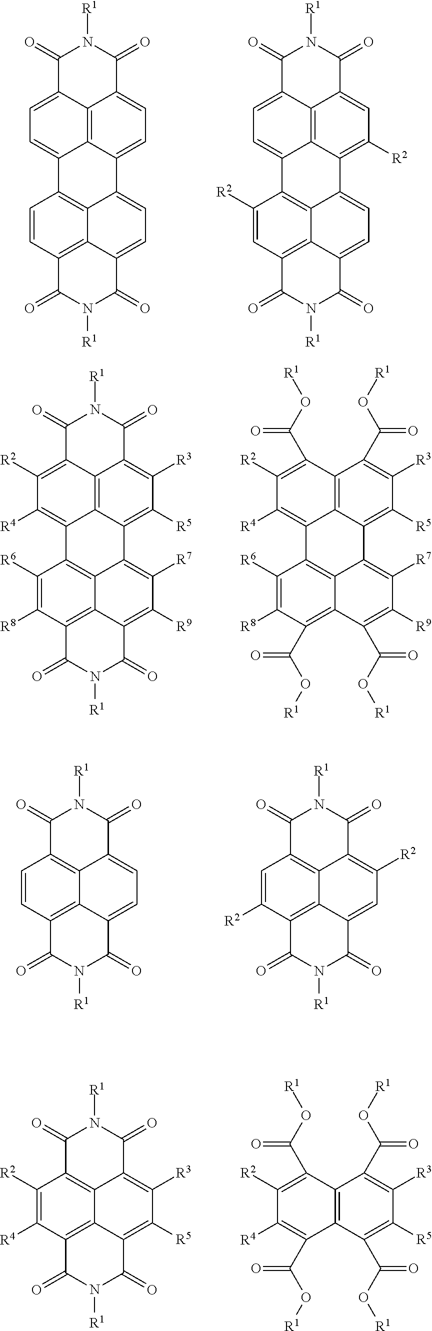 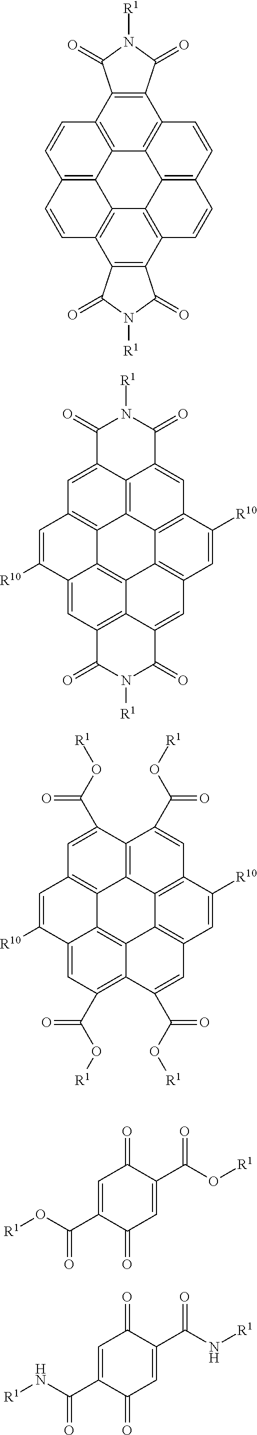
wherein:
R1 can be any of: H, polyethylene glycol (PEG); PEG-OMe, PEG-O-alkyl; PEG-O-aryl; PEG-OR; PEG-R; PEG-N₃; PEG-alkenyl; PEG-alkynyl; PEG-dye; PEG-DTPA[M]; PEG-DOTA[M]; PEG-adamantyl; PEG-CO₂H; aryl; heteroaryl; alkyl; alkenyl; alkynyl; heteroalkyl; R-PPh₃+; R—N₃; R-alkenyl; R-alkynyl; R—CO₂H; NH—R; O-alkyl; O-aryl; and, perfluoroalkyl;
R2-R9 can be any electron withdrawing group, including: halogens, including Cl, Br, F, I; CF₃; R—Cl; R—Br; R—I; R—CF₃; carbonyl; nitrile; amide; imide; cyano; carboxylic acid; carboxylate; ester; ketone; aldehyde; nitro; cyano; fluoro, chloro; bromo; iodo; sulfonate; sulfoxide, sulfone, alkyl sulfonate, sulfonic acid, alkyl sulfonates, aryl; arylamino; arylimido; arylcyano; fused/extended aryl ring systems; heteroaryl; alkyl; alkenyl; alkynyl; hetroalkyl; acyl; NO₂; NH—R; O—R; SH—R; O-alkyl; O-aryl; and, acyl-R;
R10 can be any group or combination of groups set forth in R1 to R9;
R can be any compatible functional groups; and
M can be any and all compatible metals.

3. The method of claim 1 wherein the T-cell mediated diseases include autoimmune diseases.

4. The method of claim 1 wherein the autoimmune diseases include rheumatoid arthritis, multiple sclerosis, rheumatoid arthritis, reactive arthritis, ankylosing spondylitis, systemic lupus erythematosus, glomerulonephritis, psoriasis, scleroderma, alopecia aerata, type 1 diabetes mellitus, celiac sprue disease, colitis, pernicious anemia, encephalomyelitis, vasculitis, thyroiditis, Grave's disease, Addison's disease, Sjogren's syndrome, antiphospholipid syndrome, autoimmune cardiomyopathy, autoimmune hemolytic anemia, autoimmune hepatitis, autoimmune inner ear disease, autoimmune lymphoproliferative disorder, autoimmune peripheral neuropathy, pancreatitis, polyendocrine syndrome, thrombocytopenic purpura, uveitis, Behcet's disease, narcolepsy, myositis, polychondritis, asthma, chronic obstructive pulmonary disease, graft-versus-host disease, and chronic graft rejection.

5. The method of claim 2 wherein the non-phenolic aromatic structures include the following and their pharmaceutically acceptable salts: bis-methoxyoctaethylene glycol perylene diimide, p-benzoquinone, and bis-methoxytriethylene glycol naphthalene diimide.

6. The method of claim 5 wherein the pharmaceutically acceptable salts include: (1) acid addition salts, formed with inorganic acids including hydrochloric acid, hydrobromic acid, sulfuric acid, nitric acid, phosphoric acid; or formed with organic acids including acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid; or (2) salts formed when an acidic proton present in the active ingredient either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine.

7. The method of claim 1 including racemates, racemic mixtures, and individual isomers of the active ingredient.

8. The method of claim 1 wherein the formulation is administered topically, orally, transdermally, or parenterally.

9. The method of claim 6 wherein the administration is by intramuscular, intradermal, intravenous, subcutaneous, intraosseous, intraperitoneal, intrathecal, epidural, intracardiac, intraarticular, intracavernous, or intravitreal injection.

10. The method of claim 1 wherein the formulation includes polymeric acids or mixtures of polymeric acids with one or more of: shellac, cetyl alcohol, and cellulose acetate, acting as an enteric coating.

11. The method of claim 1 wherein the formulation includes one or more of: aqueous solutions, suitably flavored syrups, aqueous or oil suspensions, and flavored emulsions with edible oils such as corn oil, cottonseed oil, sesame oil, coconut oil, and peanut oil.

12. A method of treating any of the following diseases or conditions: reactive oxygen species mediated, ischemic or reperfusion-related, or T-cell mediated, comprising:
administering a labeled compound including non-phenolic aromatic structures that are electron deficient and are capable of converting the superoxide radical to O₂; and/or of converting superoxide radical to oxygen and hydrogen peroxide, or pharmaceutically acceptable salts of said compound;
determining if the compound binds to or interacts with the superoxide radical;
administering additional dosages of the compound if it is determined to bind to or interact with the superoxide radical.

13. The method of claim 12 wherein the compound is labeled with a radioactive isotope.

14. The method of claim 12 wherein the non-phenolic aromatic structures include the following and their pharmaceutically acceptable salts: 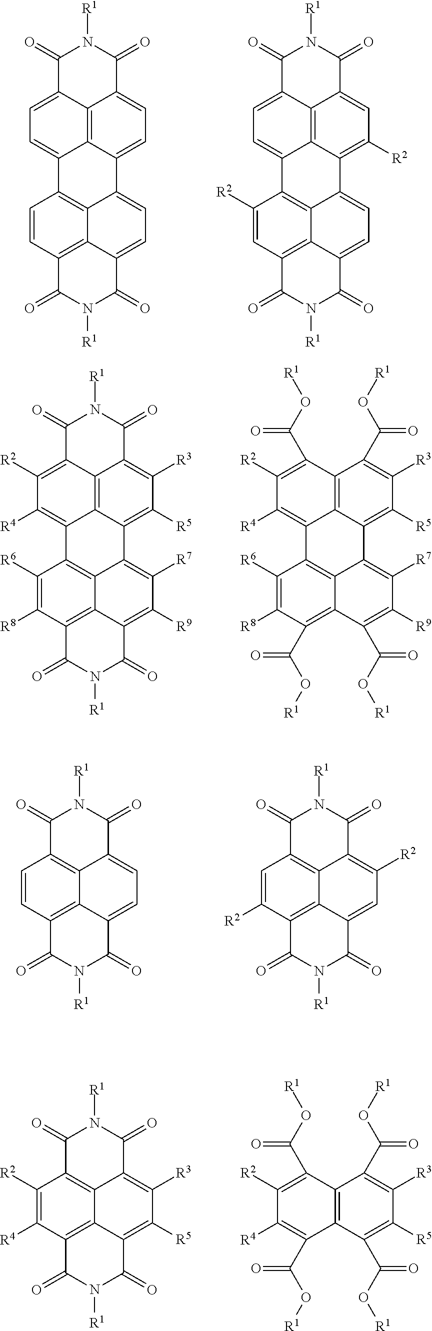 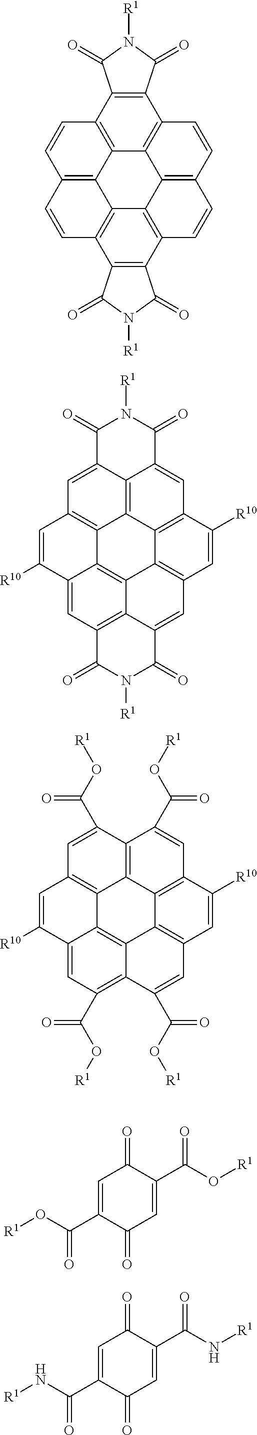
wherein:
R1 can be any of: H, polyethylene glycol (PEG); PEG-OMe, PEG-O-alkyl; PEG-O-aryl; PEG-OR; PEG-R; PEG-N₃; PEG-alkenyl; PEG-alkynyl; PEG-dye; PEG-DTPA[M]; PEG-DOTA[M]; PEG-adamantyl; PEG-CO₂H; aryl; heteroaryl; alkyl; alkenyl; alkynyl; heteroalkyl; R-PPh₃+; R—N₃; R-alkenyl; R-alkynyl; R—CO₂H; NH—R; O-alkyl; O-aryl; and, perfluoroalkyl;
R2-R9 can be any electron withdrawing group, including: halogens, including Cl, Br, F, I; CF₃; R—Cl; R—Br; R—I; R—CF₃; carbonyl; nitrile; amide; imide; cyano; carboxylic acid; carboxylate; ester; ketone; aldehyde; nitro; cyano; fluoro, chloro; bromo; iodo; sulfonate; sulfoxide, sulfone, alkyl sulfonate, sulfonic acid, alkyl sulfonates, aryl; arylamino; arylimido; arylcyano; fused/extended aryl ring systems; heteroaryl; alkyl; alkenyl; alkynyl; hetroalkyl; acyl; NO₂; NH—R; O—R; SH—R; O-alkyl; O-aryl; and, acyl-R;
R10 can be any group or combination of groups set forth in R1 to R9;
R can be any compatible functional groups; and
M can be any and all compatible metals.

15. The method of claim 12 wherein the cell mediated diseases include autoimmune diseases.

16. The method of claim 15 wherein the autoimmune diseases include rheumatoid arthritis, multiple sclerosis, systemic lupus erythomatosus, psoriasis, Graves' Disease, Addison's disease, Sjorgen's syndrome, Crohn's disease, Type 1 diabetes, scleroderma, myasthenia gravis, and fibromyalgia.

17. The method of claim 14 wherein the non-phenolic aromatic structures include the following and their pharmaceutically acceptable salts: bis-methoxyoctaethylene glycol perylene diimide, p-benzoquinone, and bis-methoxytriethylene glycol naphthalene diimide.

18. The method of claim 12 including racemates, racemic mixtures, and individual isomers of the non-phenolic aromatic structures.

19. The method of claim 12 wherein the labeled compound is administered topically, orally, transdermally, or parenterally.

20. The method of claim 19 wherein the administration is by intramuscular, intradermal, intravenous, subcutaneous, intraosseous, intraperitoneal, intrathecal, epidural, intracardiac, intraarticular, intracavernous, or intravitreal injection.
